SEQUENCE LISTING (3 sequences)

SEQ ID NO: 1 - DNA, 47 bp, Artificial
  Primer for CFTR
agggttgcgatggttctgttgtaggtaggtttggttgacttggtagg

SEQ ID NO: 2 - DNA, 27 bp, Artificial
  Probe to non-target binding primer sequence
tacctacaacagaaccatcgcaaccct

SEQ ID NO: 3 - DNA, 19 bp, Homo sapiens
gcagtgggctgtaaactcc

(19) United States
(12) Patent Application Publication
(10) Pub. No.: US 2007/0015180 A1
(43) Pub. Date: Jan. 18, 2007

(54) Oligonucleotide probe/primer compositions and methods for polynucleotide detection

(75) Inventor: Joseph A. Sorge, Del Mar, CA (US)

Correspondence Address: PALMER & DODGE, LLP;KATHLEEN M. WILLIAMS / STR, 111 HUNTINGTON AVENUE, BOSTON, MA, 02199, US

(73) Assignee: Stratagene California

(21) Appl. No.: 11/415,716

(22) Filed: May 1, 2006

Related U.S. Application Data
(60) Provisional application No. 60/676,766, filed on May 2, 2005.

(51) Int. Cl. C12Q 1/68; C07H 21/04

(52) U.S. Cl. 435/6; 536/24.3; 536/25.32

(57) ABSTRACT

The invention is related to a labeled oligonucleotide pair for detecting a target nucleic acid and methods, kits and compositions containing the labeled oligonucleotide pair. The labeled oligonucleotide pair forms a complex comprising a nucleic acid primer and a nucleic acid probe.

### RELATED APPLICATION

This application is a continuation of Application No. 60/676,766 filed May 2, 2005. The entire teachings of the above application is incorporated herein by reference.

### BACKGROUND

Techniques for polynucleotide detection have found widespread use in basic research, diagnostics, and forensics. Polynucleotide detection can be accomplished by a number of methods. Most methods rely on the use of the polymerase chain reaction (PCR) to amplify the amount of target DNA.

The TaqMan™ assay is a homogenous assay for detecting polynucleotides (see U.S. Pat. No. 5,723,591). In this assay, two PCR primers flank a central probe oligonucleotide. The probe oligonucleotide contains a fluorophore and quencher. During the polymerization step of the PCR process, the 5′ nuclease activity of the polymerase cleaves the probe oligonucleotide, causing the fluorophore moiety to become physically separated from the quencher, which increases fluorescence emission. As more PCR product is created, the intensity of emission at the novel wavelength increases. However, background emission can be rather high with this method, due to the required separation of the fluorophore and quencher in the probe oligonucleotide.

Molecular beacons are an alternative to TaqMan for the detection of polynucleotides (see U.S. Pat. Nos. 6,277,607; 6,150,097; and 6,037,130). Molecular beacons are oligonucleotide hairpins which undergo a conformational change upon binding to a perfectly matched template. The conformational change of the oligonucleotide increases the physical distance between a fluorophore moiety and a quencher moiety present on the oligonucleotide. This increase in physical distance causes the effect of the quencher to be diminished, thus increasing the signal derived from the fluorophore.

The adjacent probes method amplifies the target sequence by polymerase chain reaction in the presence of two nucleic acid probes that hybridize to adjacent regions of the target sequence, one of the probes being labeled with an acceptor fluorophore and the other probe labeled with a donor fluorophore of a fluorescence energy transfer pair. Upon hybridization of the two probes with the target sequence, the donor fluorophore interacts with the acceptor fluorophore to generate a detectable signal. The sample is then excited with light at a wavelength absorbed by the donor fluorophore and the fluorescent emission from the fluorescence energy transfer pair is detected for the determination of that target amount. U.S. Pat. No. 6,174,670B1 discloses such methods.

Sunrise primers utilize a hairpin structure similar to molecular beacons, but attached to a target binding sequence which serves as a primer. When the primer's complementary strand is synthesized, the hairpin structure is disrupted, thereby eliminating quenching. These primers detect amplified product and do not require the use of a polymerase with a 5′ exonuclease activity. Sunrise primers are described by Nazarenko et al. (Nucleic Acids Res. 25:2516-21 (1997) and in U.S. Pat. No. 5,866,336.

Scorpion probes combine a primer with an added hairpin structure, similar to Sunrise primers. However, the hairpin structure of Scorpion probes is not opened by synthesis of the complementary strand, but by hybridization of part of the hairpin structure with a portion of the target which is downstream from the portion which hybridizes to the primer.

DzyNA-PCR involves a primer containing the antisense sequence of a DNAzyme, an oligonucleotide capable of cleaving specific RNA phosphodiester bonds. The primer binds to a target sequence and drives an amplification reaction producing an amplicon which contains the active DNAzyme. The active DNAzyme then cleaves a generic reporter substrate in the reaction mixture. The reporter substrate contains a fluorophore-quencher pair, and cleavage of the substrate produces a fluorescence signal which increases with the amplification of the target sequence. Dzy-PCR is described in Todd et al., Clin. Chem. 46:625-30 (2000), and in U.S. Pat. No. 6,140,055.

Fiandaca et al. describes a fluorogenic method for PCR analysis utilizing a quencher-labeled peptide nucleic acid (Q-PNA) probe and a fluorophore-labeled oligonucleotide primer. Fiandaca et al. Genome Research. 11:609-613 (2001). The Q-PNA hybridizes to a tag sequence at the 5′ end of the primer.

Li et al. describes a double stranded probe having a quencher and fluorophore on opposite oligonucleotide strands. Li et al. Nucleic Acids Research. 30 (2e5); 1-9. When not bound to the target, the strands hybridize to each other and the probe is quenched. However, when a target is present at least one strand hybridizes to the target resulting in a fluorescent signal.

### SUMMARY OF THE INVENTION

The invention is related to novel compositions and methods for nucleic acid detection. In one aspect, the invention provides a labeled oligonucleotide pair for detecting a target nucleic acid sequence. The labeled oligonucleotide pair forms a complex having a nucleic acid primer and a nucleic acid probe. The nucleic acid primer has a first portion and a second portion. The first portion is complementary to a target nucleic acid and the second portion is complementary to a nucleic acid probe. However, the second portion is not complementary to the target nucleic acid. The nucleic acid probe is complementary to the second portion of the nucleic acid primer. However, the nucleic acid probe is not complementary to the first portion of the nucleic acid primer. The oligonucleotide probe complex also contains a pair of interactive labels. The first member of the pair of interactive labels is coupled to the nucleic acid primer and the second member is coupled to the nucleic acid probe. When the probe and primer form a complex the labels interact and when the primer and probe dissociate, the labels do not interact.

In another aspect, the invention provides methods for detecting a target nucleic acid sequence in a sample. The method involves providing to a PCR amplification reaction mixture the labeled oligonucleotide pair of the invention. Reaction conditions are applied to the PCR amplification reaction mixture which permits the cleavage of the nucleic acid probe when the target nucleic acid is present. The probe is cleaved when the cleavage enzyme contacts the probe that is hybridized to the second portion of the primer nucleic acid that has been incorporated into the amplicon. The cleavage generates a detectable signal, which is indicative of the presence of the target nucleic acid in the sample.

In a related aspect, the method for detecting a target nucleic acid sequence in a sample requires performing a PCR amplification reaction and a nuclease cleavage reaction. The PCR amplification reaction mixture includes a target nucleic acid, the labeled oligonucleotide pair and a second primer complementary to the target nucleic acid. During or after the amplification reaction the signal generated by the separation of the pair of interactive labels is detected. The signal is indicative of the presence and/or amount of the target nucleic acid sequence in the sample.

In an additional aspect of the invention, the labeled oligonucleotide pair is included in a kit. In addition to the labeled oligonucleotide pair, the kit may also include a nucleic acid polymerase, an endonuclease, a second primer, and packaging material therefor. The nucleic acid probe and nucleic acid primer of the labeled oligonucleotide pair may be supplied in either the same or separate containers within the kit.

In a final aspect of the invention, the labeled oligonucleotide pair is present in a reaction mixture for generating a signal indicative of the presence of a target nucleic acid sequence in a sample. The reaction mixture may also include a nucleic acid polymerase, a nuclease and a second primer.

### BRIEF DESCRIPTION OF THE DRAWINGS

FIG. 1 illustrates the structure of the duplex primer/probe of the invention.

FIG. 2A-2C illustrates a method of detecting a target utilizing the duplex primer/probe.

FIG. 3 shows the Q-PCR amplification plot for a detection assay utilizing 100 nM or 200 nM of the nucleic acid primer and 200 nM of the nucleic acid probe.

### DETAILED DESCRIPTION

Definitions

As used herein, a “polynucleotide” refers to a covalently linked sequence of nucleotides (i.e., ribonucleotides for RNA and deoxyribonucleotides for DNA) in which the 3′ position of the pentose of one nucleotide is joined by a phosphodiester linkage to the 5′ position of the pentose of the next nucleotide. The term “polynucleotide” includes single- and double-stranded polynucleotides. The term “polynucleotide” as it is employed herein embraces chemically, enzymatically, of metabolically modified forms of polynucleotide. “Polynucleotide” also embraces a short polynucleotide, often referred to as an oligonucleotide (e.g., a primer or a probe). A polynucleotide has a “5′-terminus” and a “3′-terminus” because polynucleotide phosphodiester linkages occur between the 5′ carbon and 3′ carbon of the pentose ring of the substituent mononucleotides. The end of a polynucleotide at which a new linkage would be to a 5′ carbon is its 5′ terminal nucleotide. The end of a polynucleotide at which a new linkage would be to a 3′ carbon is its 3′ terminal nucleotide. A “terminal nucleotide”, as used herein, is the nucleotide at the end position of the 3′- or 5′-terminus. As used herein, a polynucleotide sequence, even if internal to a larger polynucleotide (e.g., a sequence region within a polynucleotide), also can be said to have 5′- and 3′-ends.

As used herein, the term “oligonucleotide” refers to a short polynucleotide, typically less than or equal to 150 nucleotides long (e.g., between 5 and 150, preferably between 10 and 100, more preferably between 15 and 50 nucleotides in length). However, as used herein, the term is also intended to encompass longer or shorter polynucleotide chains. An “oligonucleotide” may hybridize to other polynucleotides or target nucleic acids, therefore serving as a probe for polynucleotide detection, or a primer for polynucleotide chain extension.

As used herein, a “nucleic acid primer” refers to an oligonucleotide having or containing the length limits of an “oligonucleotide” as defined above, and having or containing a sequence complementary to a target nucleic acid, which hybridizes to the target polynucleotide through base pairing so to initiate an elongation (extension) reaction to incorporate a nucleotide into the oligonucleotide primer. The nucleic acid primer contains a first and second portion, wherein the first portion is 3′ to said second portion. The first portion is complementary to and hybridizes with the target nucleic acid, and the second portion is complementary to and hybridizes with the nucleic acid probe. The nucleic acid primer is incorporated into the amplicon upon extension of the nucleic acid primer. The conditions for initiation and extension include the presence of four different deoxyribonucleoside triphosphates and a polymerization-inducing agent such as DNA polymerase or reverse transcriptase, in a suitable buffer (“buffer” includes substituents which are cofactors, or which affect pH, ionic strength, etc.) and at a suitable temperature. The nucleic acid primers useful in the present invention are generally between about 10 and 100 nucleotides in length, preferably between about 17 and 50 nucleotides in length, and most preferably between about 17 and 45 nucleotides in length.

As used herein, “nucleic acid probe” refers to an oligonucleotide, which hybridizes to the second portion of the nucleic acid primer due to complementarily of the sequence in the probe with the sequence in the second portion of the nucleic acid primer. The nucleic acid probe does not hybridize to the target nucleic acid. However, the nucleic acid probe hybridizes to the amplified target nucleic acid or amplicon after one or more cycles of amplification. The nucleic acid probe is not a peptide nucleic acid (PNA) probe. Generally, the probe comprises from 8 to 100 nucleotides, preferably from 15 to 50 nucleotides and even more preferably from 15 to 35 nucleotides.

As used herein, “labeled oligonucleotide pair” refers to a complex of two oligonucleotides: (1) the nucleic acid primer; and (2) the nucleic acid probe. The complex is formed when the nucleic acid probe hybridizes to the second portion of the nucleic acid primer. The labeled oligonucleotide pair also includes a pair of interactive labels. One member of the pair of interactive labels is coupled to the nucleic acid primer and a second member of the pair of interactive labels is coupled to the nucleic acid probe.

As used herein, a “pair of interactive labels” refers to a pair of molecules which interact physically, optically or otherwise in such a manner as to permit detection of their proximity by means of a detectable signal. Examples of a “pair of interactive labels” include, but are not limited to, labels suitable for use in fluorescence resonance energy transfer (FRET)(Stryer, L. Ann. Rev. Biochem. 47, 819-846, 1978), scintillation proximity assays (SPA) (Hart and Greenwald, Molecular Immunology 16:265-267, 1979; U.S. Pat. No. 4,658,649), luminescence resonance energy transfer (LRET) (Mathis, G. Clin. Chem. 41, 1391-1397, 1995), direct quenching (Tyagi et al., Nature Biotechnology 16, 49-53, 1998), chemiluminescence energy transfer (CRET) (Campbell, A. K., and Patel, A. Biochem. J. 216, 185-194, 1983), bioluminescence resonance eriergy transfer (BRET) (Xu, Y., Piston D. W., Johnson, Proc. Natl. Acad. Sc., 96, 151-156, 1999), or excimer formation (Lakowicz, J. R. Principles of Fluorescence Spectroscopy, Kluwer Academic/Plenum Press, New York, 1999).

As used herein, the term “complementary” refers to the concept of sequence complementarity between regions of two polynucleotide strands. It is known that an adenine base of a first polynucleotide region is capable of forming specific hydrogen bonds (“base pairing”) with a base of a second polynucleotide region which is antiparallel to the first region if the base is thymine or uracil. Similarly, it is known that a cytosine base of a first polynucleotide strand is capable of base pairing with a base of a second polynucleotide strand which is antiparallel to the first strand if the base is guanine. A first region of a polynucleotide is complementary to a second region a different polynucleotide if, when the two regions are arranged in an antiparallel fashion, at least one nucleotide of the first region is capable of base pairing with a base of the second region. Therefore, it is not required for two complementary polynucleotides to base pair at every nucleotide position. “Complementary” can refer to a first polynucleotide that is 100% or “fully” complementary to a second polynucleotide and thus forms a base pair at every nucleotide position. “Complementary” also can refer to a first polynucleotide that is not 100% complementary (e.g., 90%, 80%, 70% complementary or less) contains mismatched nucleotides at one or more nucleotide positions.

As used herein, the term “hybridization” or “binding” is used to describe the pairing of complementary (including partially complementary) polynucleotide strands, e.g., second region of the nucleic acid primer and the nucleic acid probe. Hybridization and the strength of hybridization (i.e., the strength of the association between polynucleotide strands) is impacted by many factors well known in the art including the degree of complementarity between the polynucleotides, stringency of the conditions involved, the melting temperature (Tₘ) of the formed hybrid, the presence of other components (e.g., the presence or absence of polyethylene glycol), the molarity of the hybridizing strands, and the G:C content of the polynucleotide strands.

As used herein, when one polynucleotide is said to “hybridize” to another polynucleotide, it means that there is some complementarity be tween the two polynucleotides or that the two polynucleotides form a hybrid. When one polynucleotide is said to not hybridize to another polynucleotide, it means that there is essentially no sequence complementarity between the two polynucleotides or that no hybrid forms between the two polynucleotides. In one embodiment, two complementary polynucleotides are capable of hybridizing to each other under high stringency hybridization conditions. Hybridization under stringent conditions is typically established by performing membrane hybridization (e.g., Northern hybridization) under high stringency hybridization conditions, defined as incubation with a radiolabeled probe in 5×SSC, 5× Denhardt's solution, 1% SDS at 65° C. Stringent washes for membrane hybridization are performed as follows: the membrane is washed at room temperature in 2×SSC/0.1% SDS and at 65° C. in 0.2×SSC/0.1% SDS, 10 minutes per wash, and exposed to film.

As used herein, “target nucleic acid” refers to a region of a polynucleotide of interest that is selected for extension, replication, amplification and detection. The target nucleic acid is present in a sample prior to amplification. Amplification of the target nucleic acid results in the incorporation of additional nucleic acid sequences into the amplicon, e.g., second portion of the nucleic acid primer. These additional nucleic acid sequences are not considered the target nucleic acid, but are part of the amplicon.

As used herein, “nucleic acid polymerase” refers to an enzyme that catalyzes the polymerization of nucleotides. Generally, the enzyme will initiate synthesis at the 3′-end of the primer annealed to a nucleic acid template sequence, and will proceed toward the 5′ end of the template strand. “DNA polymerase” catalyzes the polymerization of deoxyribonucleotides. Known DNA polymerases include, for example, Pyrococcus furiosus (Pfu) DNA polymerase (Lundberg et al., 1991, Gene, 108:1), E. coli DNA polymerase I (Lecomte and Doubleday, 1983, Nucleic Acids Res. 11:7505), T7 DNA polymerase (Nordstrom et al., 1981, J. Biol. Chem. 256:3112), Thermus thermophilus (Tth) DNA polyrnerase (Myers and Gelfand 1991, Biochemistry 30:7661), Bacillus stearothermophilus DNA polymerase (Stenesh and McGowan, 1977, Biochim Biophys Acta 475:32), Thermococcus litoralis (Tli) DNA polymerase (also referred to as Vent DNA polymerase, Cariello et al., 1991, Nucleic Acids Res, 19: 4193), 9° Nm DNA polymerase (discontinued product from New England Biolabs), Thermotoga maritima (Tma) DNA polymerase (Diaz and Sabino, 1998 Braz J. Med. Res, 31:1239), Thermus aquaticus (Taq) DNA polymerase (Chien et al., 1976, J. Bacteoriol, 127: 1550), Pyrococcus kodakaraensis KOD DNA polymerase (Takagi et al., 1997, Appl. Environ. Microbiol. 63:4504), JDF-3 DNA polymerase (Patent application WO 0132887), and Pyrococcus GB-D (PGB-D) DNA polymerase (Juncosa-Ginesta et al., 1994, Biotechniques, 16:820). The polymerase activity of any of the above enzyme can be determined by means well known in the art. One unit of DNA polymerase activity, according to the subject invention, is defined as the amount of enzyme which catalyzes the incorporation of 10 nmoles of total dNTPs into polymeric form in 30 minutes at optimal temperature (e.g., 72° C. for Pfu DNA polymerase).

As used herein, “polymerase chain reaction” or “PCR” or “PCR assay” refers to an in vitro method for amplifying a specific polynucleotide template sequence. The PCR reaction involves a repetitive series of temperature cycles and is typically performed in a volume of 50-100 □l. The reaction mix comprises dNTPs (each of the four deoxyribonucleotides dATP, dCTP, dGTP, and dTTP), primers, buffers, DNA polymerase, and polynucleotide template. One PCR reaction may consist of 5 to 100 “cycles” of denaturation and synthesis of a polynucleotide molecule. The PCR process is described in U.S. Pat. Nos. 4,683,195 and 4,683,202, the disclosures of which are incorporated herein by reference.

As used herein a “nuclease” or a “cleavage agent” refers to an enzyme that is specific for, that is, cleaves a “cleavage structure” according to the invention and is not specific for, that is, does not substantially cleave a probe that is not hybridized to a target nucleic acid. The terms nuclease or cleavage agent include an enzyme that possesses 5′ endonucleolytic activity for example a DNA polymerase, e.g. DNA polymerase I from E: coli, and DNA polymerase from Thermus aquaticus (Taq), Thermus thermophilus (Tth), Pyrococcus furiosus (Pfu) and Thermus flavus (Tfl). The terms nuclease or cleavage agent also embodies FEN nucleases. The terms nuclease or cleavage agent also include an enzyme that possesses exonuclease activity.

As used herein, a “cleavage structure” refers to a structure which is formed by the interaction of a nucleic acid probe and a target nucleic acid to form a duplex. The duplex is then cleavable by a cleavage agent.

As used herein, “cleavage reaction” refers to enzymatically separating an oligonucleotide (i.e. not physically linked to other fragments or nucleic acids by phosphodiester bonds) into fragments or nucleotides and fragments that are released from the oligonucleotide. For example, cleaving a labeled cleavage structure refers to separating a labeled cleavage structure according to the invention, into distinct fragments including fragments derived from an oligonucleotide, e.g. nucleic acid probe, that specifically hybridizes with a target, e.g., second portion of the nucleic acid primer, wherein one of the distinct fragments is a labeled nucleic acid fragment derived from an oligonucleotide e.g. nucleic acid probe, that specifically hybridizes with a target e.g., second portion of the nucleic acid primer, that can be detected and/or measured by methods well known in the art that are suitable for detecting the labeled moiety that is present on a labeled fragment. A cleavage reaction is performed by an exonuclease activity or an endonuclease activity. Cleavage reactions utilizing an endonuclease activity are described in U.S. Pat. Nos. 6,548,250, 5,210,015 and 6,528,254, which are herein incorporated by reference in their entirety. Cleavage reaction assays encompassed by the present methods also include assays utilizing exonuclease activity such as the TaqMan assay described in U.S. Pat. No. 5,723,591, which is herein incorporated by reference in its entirety. These approaches have employed probes containing fluorescence-quencher pairs where the probe is cleaved during amplification to release a fluorescent molecule whose concentration is proportional to the amount of double-stranded DNA present. During amplification, the probe is digested by the nuclease activity of a polymerase or a separate nuclease when hybridized to the target sequence. Cleavage causes the fluorescent molecule to be separated from the quencher molecule, thereby causing fluorescence from the reporter molecule to appear.

As used herein, “endonuclease” refers to an enzyme that cleaves bonds, preferably phosphodiester bonds, within a nucleic acid molecule. An endonuclease can be specific for single stranded or double-stranded DNA or RNA. An endonuclease enzyme includes for example a DNA polymerase, e.g. DNA polymerase I from E. coli, and DNA polymerase from Thermus aquaticus (Taq), Thermus thermophilus (Tth) and Thermus flavus (Tfl). The term endonuclease also embodies FEN nuclease.

As used herein, “exonuclease” refers to an enzyme that cleaves bonds, preferably phosphodiester bonds, between nucleotides one at a time from the end of a polynucleotide. An exonuclease can be specific for the 5′ or 3′ end of a DNA or RNA molecule, and is referred to herein as a 5′ exonuclease or a 3′ exonuclease.

As used herein, “5′ to 3′ exonuclease activity” or “5′→3′ exonuclease activity” refers to that activity of a template-specific nucleic acid polymerase e.g. a 5′→3′ exonuclease activity traditionally associated with some DNA polymerases whereby mononucleotides or oligonucleotides are removed from the 5′ end of a polynucleotide in a sequential manner, (i.e., E. coli DNA polymerase I has this activity whereas the Klenow (Klenow et al., 1970, Proc. Natl. Acad. Sci., USA, 65:168) fragment does not, (Klenow et al., 1971, Eur. J. Biochem., 22:371)), or polynucleotides are removed from the 5′ end by an endonucleolytic activity that may be inherently present in a 5′ to 3′ exonuclease activity.

As used herein, the phrase “substantially lacks 5′ to 3′ exonuclease activity” or “substantially lacks 5′→3′ exonuclease activity” means having less than 10%, 5%, 1%, 0.5%, or 0.1% of the activity of a wild type enzyme. The phrase “lacking 5′ to 3′exonuclease activity” or “lacking 5′→3′ exonuclease activity” means having undetectable 5′ to 3′ exonuclease activity or having less than about 1%, 0.5%, or 0.1% of the 5′ to 3′ exonuclease activity of a wild type enzyme. 5′ to 3′ exonuclease activity may be measured by an exonuclease assay which includes the steps of cleaving a nicked substrate in the presence of an appropriate buffer, for example 10 mM Tris-HCl (pH 8.0), 10 mM MgCl₂ and 50 μg/ml bovine serum albumin) for 30 minutes at 60° C., terminating the cleavage reaction by the addition of 95% formamide containing 10 mM EDTA and 1 mg/ml bromophenol blue, and detecting nicked or un-nicked product.

Nucleic acid polymerases useful in certain embodiments of the invention substantially lack 5′ to 3′ exonuclease activity and or 3′ to 5′ exonuclease activity and include but are not limited to exo—Pfu DNA polymerase (a mutant form of Pfu DNA polymerase that substantially lacks 3′ to 5′ exonuclease activity, Cline et al., 1996, Nucleic Acids Research, 24: 3546; U.S. Pat. No. 5,556,772; commercially available from Stratagene, La Jolla, Calif. Catalogue #600163), exo—Tma DNA polymerase (a mutant form of Tma DNA polymerase that substantially lacks 3′ to 5′ exonuclease activity), exo—Tli DNA polymerase (a mutant form of Tli DNA polymerase that substantially lacks 3′ to 5′ exonuclease activity New England Biolabs, (Cat #257)), exo—E. coli DNA polymerase (a mutant form of E. coli DNA polymerase that substantially lacks 3′ to 5′ exonuclease activity) exo—Klenow fragment of E. coli DNA polymerase I (Stratagene, Cat #600069), exo—T7 DNA polymerase (a mutant form of T7 DNA polymerase that substantially lacks 3′ to 5′ exonuclease activity), exo—KOD DNA polymerase (a mutant form of KOD DNA polymerase that substantially lacks 3′ to 5′ exonuclease activity), exo—JDF-3 DNA polymerase (a mutant form of JDF-3 DNA polymerase that substantially lacks 3′ to 5′ exonuclease activity), exo—PGB-D DNA polymerase (a mutant form of PGB-D DNA polymerase that substantially lacks 3′ to 5′ exonuclease activity) New England Biolabs, Cat. #259, Tth DNA polymerase, Taq DNA polymerase (e.g., Cat. Nos. 600131, 600132, 600139, Stratagene); UlTma (N-truncated) Thermatoga martima DNA polymerase; Klenow fragment of DNA polymerase I, 9°Nm DNA polymerase (discontinued product from New England Biolabs, Beverly, Mass.), “3′-5′ exo reduced” mutant (Southworth et al., 1996, Proc. Natl. Acad. Sci 93:5281) and Sequenase (USB, Cleveland, Ohio.). The polymerase activity of any of the above enzyme can be defined by means well known in the art. One unit of DNA polymerase activity, according to the subject invention, is defined as the amount of enzyme which catalyzes the incorporation of 10 nmoles of total dNTPs into polymeric form in 30 minutes at optimal temperature.

As used herein, the phrase “substantially lacks endonuclease activity” means having less than 10%, 5%, 1%, 0.5%, or 0.1% of the activity of a wild type enzyme. Endonuclease activity may be measured by a variety of endonuclease assays known in the art, including those descibed. in U.S. Pat. No. 6,548,250, which is herein incorporated by reference.

Description

The inventors have discovered a highly effective labeled oligonucleotide complex for the detection of a target nucleic acid sequence, for example in real time PCR analysis. In one aspect, the invention provides a labeled oligonucleotide pair for detecting a target nucleic acid sequence. The labeled oligonucleotide pair forms a complex having a nucleic acid primer and a nucleic acid probe. The nucleic acid primer is divided into a first portion and a second portion. The first portion is complementary to a target nucleic acid and the second portion is complementary to a nucleic acid probe. However, the second portion is not complementary to the target nucleic acid. The nucleic acid probe is complementary to the second portion of the nucleic acid primer. However, the nucleic acid probe is not complementary to the first portion of the nucleic acid primer. The oligonucleotide probe complex also contains a pair of interactive labels. The first member of the pair of interactive labels is coupled to the nucleic acid primer and the second member is coupled to the nucleic acid probe. When the probe and primer form a complex the labels interact and when the primer and probe are dissociated, the labels do not interact. In some embodiments, the pair of interactive labels is a fluorophore and a quencher. When nucleic acid probe and nucleic acid primer are hybridized to each other the labels are in sufficient proximity such that the labels interact. Preferably, one member of the interactive pair of labels is attached to the 3′ end of the nucleic acid probe and the other member is attached to the 5′ end of the nucleic acid primer. In a further prefered embodiment, one member of the interactive pair of labels is attached to the hydroxyl group of the 3′ terminal nucleotide. In one embodiment, the fluorophore is attached to the 3′ end of the nucleic acid probe and the quencher is attached to the 5′ end of the nucleic acid primer. Fluorophore useful in the invention include: FAM, R110, TAMRA, R6G, CAL Fluor Red 610, CAL Fluor Gold 540, and CAL Fluor Orange 560, Quasar 670. Quenchers useful in the invention include: DABCYL, BHQ-1, BHQ-2, and BHQ-3. In some embodiments, the fluorescence signal increases by at least three fold upon cleavage of the nucleic acid probe. In other embodiments, the fluorescent signal increases by at least four fold upon cleavage of the nucleic acid probe.

In another aspect, the invention provides methods for detecting a target nucleic acid sequence in a sample. The method involves providing to a PCR amplification reaction mixture the labeled oligonucleotide pair of the invention. Reaction conditions are applied to the PCR amplification reaction mixture which permits the cleavage of the nucleic acid probe when the target nucleic acid is present. The probe is cleaved when the cleavage enzyme comes into contact with the probe that is hybridized to the second portion of the nucleic acid primer that was incorporated into an amplicon. The cleavage generates a detectable signal, which is indicative of the presence of the target nucleic acid in the sample. In some embodiments, the method further includes providing a nucleic acid polymerase. The polymerase may substantially lack a 5′ to 3′ exonuclease and/or endonuclease activity. In another embodiment, the method further includes providing a nuclease. The nuclease may be an exonuclease or endonuclease. In some embodiments, the nuclease is FEN. In a preferred embodiment, Pfu DNA polymerase substantially lacking a 5′ to 3′ nuclease activity and FEN nuclease are provided to the reaction mixture.

In a related aspect, the method for detecting a target nucleic acid sequence in a sample requires performing a PCR amplification reaction and a nuclease cleavage reaction. The PCR amplification reaction mixture includes a target nucleic acid, the labeled oligonucleotide pair and a second primer complementary to the target nucleic acid. During or after the amplification reaction the signal generated by the separation of the pair of interactive labels is detected. The signal is indicative of the presence and/or amount of the target nucleic acid present in the sample. In some embodiments, the method further includes providing a nucleic acid polymerase. The polymerase may substantially lack a 5′ to 3′ exonuclease and/or endonuclease activity. In another embodiment, the method further includes providing a nuclease. The nuclease may be an exonuclease or endonuclease. In some embodiments, the nuclease is FEN. In a preferred embodiment, Pfu DNA polymerase substantially lacking a 5′ to 3′ nuclease activity and FEN nuclease are provided to the reaction mixture. In some embodiments, the nuclease cleavage reaction is performed by an exonuclease. In another embodiment, the nuclease cleavage reaction is performed by an endonuclease. In yet another embodiment, the nuclease cleavage reaction comprise the steps of displacing the hybridized nucleic acid probe by an extension reaction with a DNA polymerase and cleavage of the displaced strand by an endonuclease. In yet another embodiment, the nucleic acid probe is cleaved by the extension of the primer by a DNA polymerase having exonuclease activity.

In an additional aspect of the invention, the labeled oligonucleotide pair is included in a kit. In addition to the labeled oligonucleotide pair, the kit may also include a nucleic acid polymerase, an endonuclease, a second primer, and packaging material therefor. The nucleic acid probe and nucleic acid primer of the labeled oligonucleotide pair may be supplied in the kit in either the same or separate containers. In some embodiments, the kit further includes a nucleic acid polymerase. The polymerase may substantially lack a 5′ to 3′ exonuclease and/or endonuclease activity. In another embodiment, the kit further includes a nuclease. The nuclease may be an exonuclease or endonuclease. In yet another embodiment, the nuclease is FEN. In a preferred embodiment, Pfu DNA polymerase substantially lacking a 5′ to 3′ nuclease activity and the FEN nuclease are contained in the kit.

In a final aspect of the invention, the oligonucleotide complex is part of a reaction mixture for generating a signal indicative of the presence of a target nucleic acid sequence in a sample. The reaction mixture may also include a nucleic acid polymerase, a nuclease and a second primer. In some embodiments, the polymerase substantially lacks a 5′ to 3′ exonuclease and/or endonuclease activity. In another embodiment, the reaction mixture further includes a nuclease. The nuclease may be an exonuclease or endonuclease. In some embodiments, the nuclease is FEN. In still another embodiment, the DNA polymerase is Pfu DNA polymerase substantially lacking a 5′ to 3′ nuclease activity and the nuclease is FEN nuclease.

Preparation of Primers and Probes

Probes and primer can be synthesized by any method described below and other methods known in the art. Probes and primers are typically prepared by biological or chemical synthesis, although they can also be prepared by biological purification or degradation, e.g., endonuclease digestion. For short sequences such as the nucleic acid probes and primers used in the present invention, chemical synthesis is frequently more economical as compared to biological synthesis. For longer sequences standard replication methods employed in molecular biology can be used such as the use of M13 for single stranded DNA as described by Messing, 1983, Methods Enzymol. 101:20-78. Chemical methods of polynucleotide or oligonucleotide synthesis include phosphotriester and phosphodiester methods (Narang, et al., Meth. Enzymol. (1979) 68:90) and synthesis on a support (Beaucage, et al., Tetrahedron Letters. (1981) 22:1859-1862) as well as phosphoramidate technique, Caruthers, M. H., et al., Methods in Enzymology (1988)154:287-314 (1988), and others described in “Synthesis and Applications of DNA and RNA,” S. A. Narang, editor, Academic Press, New York, 1987, and the references contained therein.

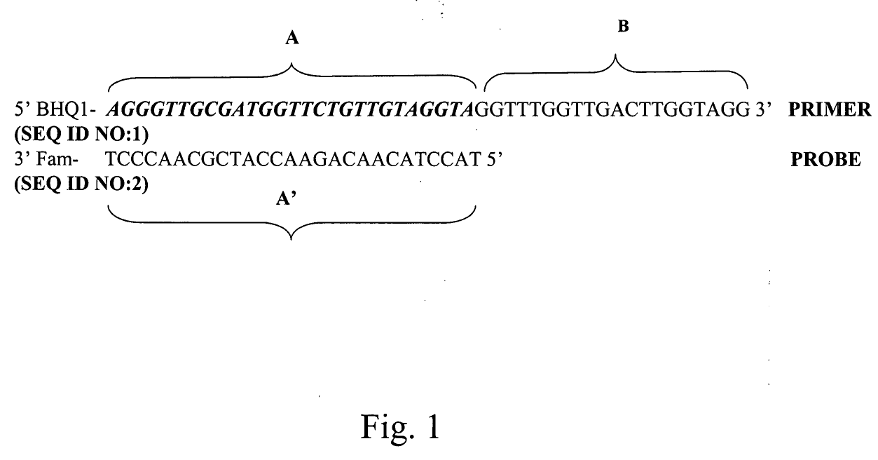

The nucleic acid probes and primers of the invention can be formed from a single strand. The labeled oligonucleotide pair containing a complex of the nucleic acid probe and nucleic acid primer can be formed from two single strands, e.g., nucleic acid probe and nucleic acid primer, which associate, for example by hybridization of complementary bases, to form the complex. See FIG. 1. The nucleic acid probe and primer can be provided so as to form a complex prior to or during an amplification reaction. For example, the nucleic acid probe and nucleic acid primer can be combined within the same reaction tube, prior to amplification. Heat can then be applied to the reaction tube so as to denature the nucleic acid probe and primer. The reaction mixture can then be cooled to allow annealing of the complementary portions of the nucleic acid probe and primer so that a complex is formed. Alternatively the nucleic acid probe and primer can be added to the amplification reaction mixture and the complex formed therein during the thermal cycling reaction.

Labels can be attached at any position on any strand, provided that a detectable signal is quenched when the nucleic acid probe hybridizes to the second portion of the nucleic acid primer and a signal is produced when the probe is cleaved in a cleavage reaction.

According to the present invention, the nucleic acid primer can comprise natural, non-natural nucleotides and analogs. The nucleic acid primer may be a nucleic acid analog or chimera comprising nucleic acid and nucleic acid analog monomer units, such as 2-aminoethylglycine. For example, part or all of the nucleic acid primer may be PNA or a PNA/DNA chimera. Oligonucleotides with minor groove binders (MGBs), locked nucleic acids (LNA) and other modified nucleotides can be used. These oligonucleotides using synthetic nucleotides can have the advantage that the length can be shortened while maintaining a high melting temperature.

The nucleic acid primer contains a first (B) and second portion (A), wherein the first portion is 3′ to the second portion. See FIG. 1. The first portion is complementary to and hybridizes with the target nucleic acid, and the second portion is complementary to and hybridizes with the nucleic acid probe (A′). The nucleic acid primer is incorporated into the amplicon upon extension of the nucleic acid primer. The second portion of the nucleic acid primer should not hybridize to the target nucleic acid. The second portion may comprise a tag sequence, e.g., GBS nucleic acid sequence, which is universal to all nucleic acid primers. The nucleic acid primers useful in the present invention are generally between about 10 and 100 nucleotides in length, preferably between about 17 and 50 nucleotides in length, and most preferably between about 17 and 45 nucleotides in length.

In one embodiment, the first and second portion of the nucleic acid primer are adjacent. In another embodiment, there is an intervening nucleic acid sequence between the first portion and the second portion of the nucleic acid primer. The intervening sequence may comprise a nucleic acid sequence that is non-complementary to the target nucleic acid and non-complementary to the nucleic acid probe. The intervening sequence is generally between about 1 and 20 nucleotides in length, preferably between about 2 and 15 nucleotides, and most preferably between about 3 and 10 nucleotides in length.

According to the present invention, the nucleic acid probe can comprise natural, non-natural nucleotides and analogs. The nucleic acid probe may be a nucleic acid analog or chimera comprising nucleic acid and nucleic acid analog monomer units, such as 2-aminoethylglycine. However, the nucleic acid probe is not PNA or a PNA/DNA chimera: Oligonucleotides with minor groove binders (MGBs), locked nucleic acids (LNA) and other modified nucleotides can be used.

The nucleic acid probe hybridizes to the second portion of the nucleic acid primer. The nucleic acid probe does not hybridize to the target nucleic acid. However, the nucleic acid probe hybridizes to the amplified target nucleic acid or amplicon after one or more cycles of amplification. The nucleic acid probe may contain a universal sequence complementary to the second portion of the nucleic acid primer. Thus, the same probe may be used in the detection of multiple different target nucleic acids. Generally, the probe comprises from 8 to 100 nucleotides, preferably from 15 to 50 nucleotides and even more preferably from 15 to 35 nucleotides.

Labels

As used herein, the phrase “interactive pair of labels” as well as the phrase “pair of interactive labels” as well as the phrase “first member and second member” refer to a pair of molecules which interact physically, optically, or otherwise in such a manner as to permit detection of their proximity by means of a detectable signal. Examples of a “pair of interactive labels” include, but are not limited to, labels suitable for use in fluorescence resonance energy transfer (FRET) (Stryer, L. Ann. Rev. Biochem. 47, 819-846, 1978), scintillation proximity assays (SPA) (Hart and Greenwald, Molecular Immunology 16:265-267, 1979; U.S. Pat. No. 4,658,649), luminescence resonance energy transfer (LRET) (Mathis, G. Clin. Chem. 41, 1391-1397, 1995), direct quenching (Tyagi et al., Nature Biotechnology 16, 49-53, 1998), chemiluminescence energy transfer (CRET) (Campbell, A. K., and Patel, A. Biochem. J. 216, 185-194, 1983), bioluminescence resonance energy transfer (BRET) (Xu, Y., Piston D. W., Johnson, Proc. Natl. Acad. Sc., 96, 151-156, 1999), or excimer formation (Lakowicz, J. R. Principles of Fluorescence Spectroscopy, Kluwer Academic/Plenum Press, New York, 1999).

The pair of labels can be either covalently or non-covalently attached to the oligonucleotide probe of the invention. Preferred are labels which are covalently attached at or near the 5′ and 3′ ends of the probe.

In one embodiment, one member of the interactive pair of labels is attached to the 3′ end of the nucleic acid probe and the other member is attached to the 5′ end of the nucleic acid primer. In another embodiment, the fluorophore is attached to the 3′ end of the nucleic acid probe and the quencher is attached to the 5′ end of the nucleic acid primer. In yet another embodiment, the fluorophore or quencher is internally attached to the nucleic acid probe or primer. In still yet another embodiment, the fluorophore and quencher are both attached to the probe.

As used herein, references to “fluorescence” or “fluorescent groups” or “fluorophores” include luminescence and luminescent groups, respectively.

An “increase in fluorescence”, as used herein, refers to an increase in detectable fluorescence emitted by a fluorophore. An increase in fluorescence may result, for example, when the distance between a fluorophore and a quencher is increased, for example due to the cleavage of the probe by a nuclease, such that the quenching is reduced. There is an “increase in fluorescence” when the fluorescence emitted by the fluorophore is increased by at least 2 fold, for example 2, 2.5, 3, 4, 5, 6, 7, 8, 10 fold or more. Cleavage, for example by a 5′-flap endonuclease (FEN) or other nuclease, can be used to separate the first and second labels and thus to enhance the signal produced by binding to target.

Fluorophores

A pair of interactive labels useful for the invention can comprise a pair of FRET-compatible dyes, or a quencher-dye pair. In one embodiment, the pair comprises a fluorophore-quencher pair.

The oligonucleotide pair complex of the present invention permits monitoring of amplification reactions by fluorescence. They can be labeled with a fluorophore and quencher in such a manner that the fluorescence emitted by the fluorophore in an intact complex is substantially quenched, whereas the fluorescence in a complex where the nucleic acid probe has been cleaved is not quenched, resulting in an increase in overall fluorescence upon probe cleavage. Furthermore, the generation of a fluorescent signal during real-time detection of the amplification products allows accurate quantitation of the initial number of target sequences in a sample.

A wide variety of fluorophores can be used, including but not limited to: 5-FAM (also called 5-carboxyfluorescein; also called Spiro(isobenzofuran-1(3H), 9′-(9H)xanthene)-5-carboxylic acid,3′,6′-dihydroxy-3-oxo-6-carboxyfluorescein); 5-Hexachloro-Fluorescein ([4,7,2′,4′,5′,7′-hexachlor-(3′,6′-dipivaloyl-fluoresceinyl)-6-carboxylic acid]); 6-Hexachloro-Fluorescein([4,7,2′,4′,5′,7′-hexachloro-(3′,6′-dipivaloylfluoresceinyl)-5-carboxylic acid]); 5-Tetrachloro-Fluorescein ([4,7,2′,7′-tetra-chloro-(3′,6′-dipivaloylfluoresceinyl)-5-carboxylic acid]); 6-Tetrachloro-Fluorescein ([4,7,2′,7′-tetrachloro-(3′,6′-dipivaloylfluoresceinyl)-6-carboxylic acid]); 5-TAMRA (5-carboxytetramethylrhodamine; Xanthylium, 9-(2,4-dicarboxyphenyl)-3,6-bis(dimethyl-amino); 6-TAMRA (6-carboxytetramethylrhodamine; Xanthylium, 9-(2,5-dicarboxyphenyl)-3,6-bis(dimethylamino); EDANS (5-((2-aminoethyl) amino)naphthalene-1-sulfonic acid); 1,5-IAEDANS (5-((((2-iodoacetyl)amino)ethyl) amino)naphthalene-1-sulfonic acid); DABCYL (4-((4-(dimethylamino)phenyl) azo)benzoic acid) Cy5(Indodicarbocyanine-5) Cy3 (Indo-dicarbocyanine-3); and BODIPY FL (2,6-dibromo-4,4-difluoro-5,7-dimethyl-4-bora-3a,4a-diaza-s-indacene-3-proprionic acid), Quasar-670 (Biosearch Technologies), CalOrange (Biosearch Technologies), Rox, as well as suitable derivatives thereof.

Quenchers

As used herein, the term “quencher” refers to a chromophoric molecule or part of a compound, which is capable of reducing the emission from a fluorescent donor when attached to or in proximity to the donor. Quenching may occur by any of several mechanisms including fluorescence resonance energy transfer, photo-induced electron transfer, paramagnetic enhancement of intersystem crossing, Dexter-exchange coupling, and exciton coupling such as the formation of dark complexes. Fluorescence is “quenched” when the fluorescence emitted by the fluorophore is reduced as compared with the fluorescence in the absence of the quencher by at least 10%, for example, 15%, 20%, 30%, 40%, 50%, 60%, 70%, 80%, 90%, 95%, 98%, 99%, 99.9% or more.

The quencher can be any material that can quench at least one fluorescence emission from an excited fluorophore being used in the assay. There is a great deal of practical guidance available in the literature for selecting appropriate reporter-quencher pairs for particular probes, as exemplified by the following references: Clegg (1993, Proc. Natl. Acad. Sci., 90:2994-2998); Wu et al. (1994, Anal. Biochem., 218:1-13); Pesce et al., editors, Fluorescence Spectroscopy (1971, Marcel Dekker, New York); White et al., Fluorescence Analysis: A Practical Approach (1970, Marcel Dekker, New York); and the like. The literature also includes references providing exhaustive lists of fluorescent and chromogenic molecules and their relevant optical properties for choosing reporter-quencher pairs, e.g., Berlman, Handbook of Fluorescence Spectra of Aromatic Molecules, 2nd Edition (1971, Academic Press, New York); Griffiths, Colour and Constitution of Organic Molecules (1976, Academic Press, New York); Bishop, editor, Indicators (1972, Pergamon Press, Oxford); Haugland, Handbook of Fluorescent Probes and Research Chemicals (1992 Molecular Probes, Eugene) Pringsheim, Fluorescence and Phosphorescence (1949, Interscience Publishers, New York), all of which incorporated hereby by reference. Further, there is extensive guidance in the literature for derivatizing reporter and quencher molecules for covalent attachment via common reactive groups that can be added to an oligonucleotide, as exemplified by the following references, see, for example, Haugland (cited above); Ullman et al., U.S. Pat. No. 3,996,345; Khanna et al., U.S. Pat. No. 4,351,760, all of which hereby incorporated by reference.

A number of commercially available quenchers are known in the art, and include but are not limited to DABCYL, BHQ-1, BHQ-2, and BHQ-3. The BHQ (“Black Hole Quenchers”) quenchers are a new class of dark quenchers that prevent fluorescence until a hybridization event occurs. In addition, these new quenchers have no native fluorescence, virtually eliminating background problems seen with other quenchers. BHQ quenchers can be used to quench almost all reporter dyes and are commercially available, for example, from Biosearch Technologies, Inc (Novato, Calif.).

Attachment of Fluorophore and Quencher

In one embodiment of the invention, the fluorophore or quencher is attached to the 3′ nucleotide of the nucleic acid probe or nucleic acid primer. In another embodiment of the invention, the fluorophore or quencher is attached to the 5′ nucleotide. In yet another embodiment, the fluorophore or quencher is internally attached to the nucleic acid probe or primer. In still yet another embodiment, the fluorophore and quencher are both attached to the nucleic acid probe. In another embodiment, one of said fluorophore or quencher is attached to the 5′ nucleotide of either the nucleic acid probe or nucleic acid primer and the other of said fluorophore or quencher is attached to the 3′ nucleotide of the other. In a preferred embodiment, the fluorophore is attached to the 3′ nucleotide of the nucleic acid probe and the quencher is attached to the 5′ nucleotide of the nucleic acid primer. Attachment can be made via direct coupling, or alternatively using a spacer molecule of between 1 and 5 atoms in length.

For the internal attachment of the fluorophore or quencher, linkage can be made using any of the means known in the art. Appropriate linking methodologies for attachment of many dyes to oligonucleotides are described in many references, e.g., Marshall, Histochemical J., 7: 299-303 (1975); Menchen et al., U.S. Pat. No. 5,188,934; Menchen et al., European Patent Application 87310256.0; and Bergot et al., International Application PCT/US90/05565. All are hereby incorporated by reference.

Each member of the fluorophore/quencher pair can be attached anywhere within the nucleic acid probe or primer, preferably at a distance from the other of the pair such that sufficient amount of quenching occurs when the nucleic acid probe and primer are hybridized.

When the labeled oligonucleotide pair forms a complex, the moieties of the fluorophore/quencher pair are in a close, quenching relationship. For maximal quenching, the two moieties are ideally close to each other. In one embodiment, the quencher and fluorophore are positioned 30 or fewer nucleotides from each other.

Preferably the labeled oligonucleotide pair is used to monitor or detect the presence of a target DNA in a nucleic acid amplification reaction. The method, according to the invention, is performed using typical reaction conditions for standard polymerase chain reaction (PCR). Two temperatures are achieved per cycle: one, a high temperature denaturation step (generally between 90° C. and 96° C.), typically between 1 and 30 seconds, and a combined annealing/extension step (anywhere between 50° C. and 65° C., depending on the annealing temperature of the probe and primer), usually between 10 and 90 seconds. The reaction mixture, also referred to as the “PCR reaction mixture” or “PCR mixture”, may contain a nucleic acid, a nucleic acid polymerase as described above, the labeled oligonucleotide pair of the present invention, a second primer, suitable buffer, and salts. The reaction can be performed in any thermal-cycler commonly used for PCR. However, preferred are cyclers with real-time fluorescence measurement capabilities, including instruments capable of measuring real-time including Taq Man 7700 AB (Applied Biosystems, Foster City, Calif.), Rotorgene 2000 (Corbett Research, Sydney, Australia), LightCycler (Roche Diagnostics Corp, Indianapolis, Ind.), iCycler (Biorad Laboratories, Hercules, Calif.), Mx3OOOP Real-Time PCR System, Mx3005P Real-Time PCR System (Stratagene, La Jolla, Calif.) and Mx4000 Multi-Plex Quantitative PCR System (Stratagene, La Jolla, Calif.).

Use of a labeled probe generally in conjunction with the amplification of a target polynucleotide, for example, by PCR, e.g., is described in many references, such as Innis et al., editors, PCR Protocols (Academic Press, New York, 1989); Sambrook et al., Molecular Cloning, Second Edition (Cold Spring Harbor Laboratory, New York, 1989), all of which are hereby incorporated herein by reference.

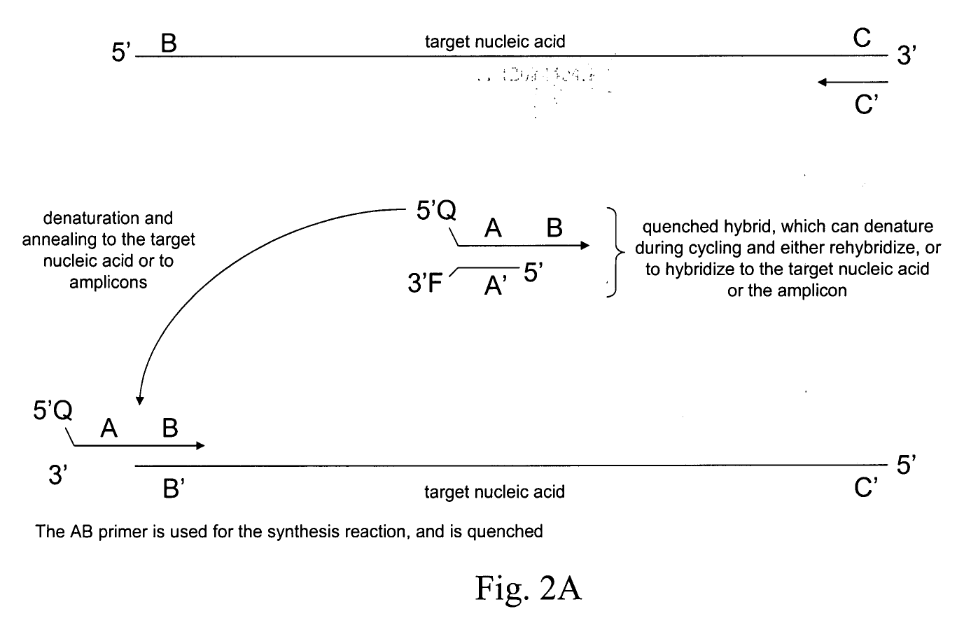

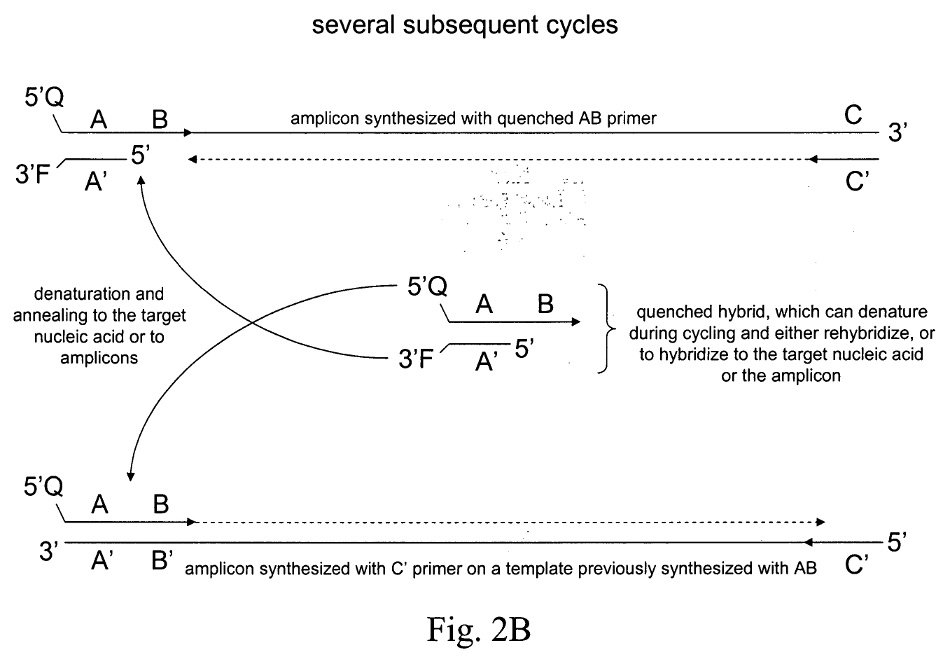

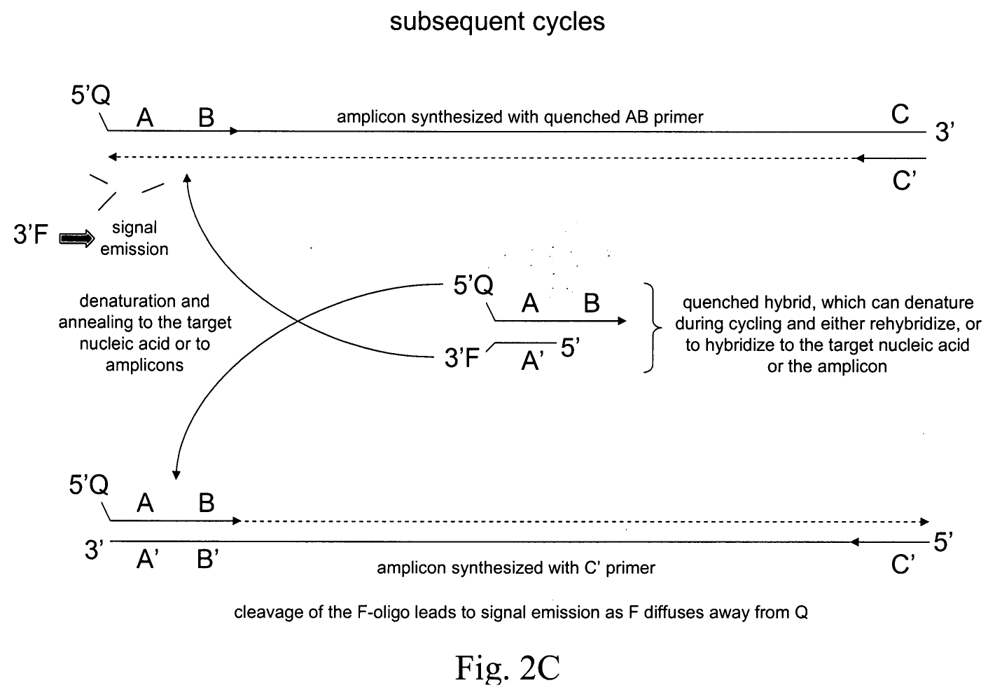

In the present invention the nucleic acid primer and nucleic acid probe are added to a PCR reaction mixture separately or as a complex. When added as a complex the quencher inhibits the fluorescent signal. FIG. 2A. During the denaturing step the probe and primer separate. FIG. 2A. In the first annealing step, the nucleic acid primer reanneals to the nucleic acid probe or anneals to the target nucleic acid via the first portion of the primer, while the second primer anneals to the complement of the target nucleic acid. During the extension step, the nucleic acid primer is extended by the DNA polymerase, thus synthesizing an anplicon which is complementary to the target nucleic acid and incorporating the second portion of the primer nucleic acid. The second primer is also extended. The reaction is repeated for additional cycles.

In the subsequent cycles the oligonucleotide complex is again denatured and may reanneal or the primer nucleic acid may anneal to the target via its first portion. Alternatively, both the first and second portions of the nucleic acid primer may anneal to the amplicon which has incorporated the second portion of the nucleic acid primer. FIG. 2B. The second primer may anneal to the amplicon having the incorporated nucleic acid primer, while the nucleic acid probe hybridizes to the second portion of the nucleic acid primer that has been incorporated into the amplicon. The DNA polymerase extends the second primer into the region occupied by the annealed labeled nucleic acid probe. FIG. 2C. The probe is then cleaved directly by the nuclease activity of the polymerase, thus releasing the fluorescent label from the nucleic acid probe and generating a fluorescent signal.

Alternatively, the fluorescent signal is generated upon extension of the second primer by a DNA polymerase lacking nuclease activity. The DNA polymerase partially displaces the nucleic acid probe. The partial displacement of the 5′ end of the probe creates a cleavage structure which can be cleaved by a nuclease which recognizes the 5′ flap, e.g., FEN nuclease.

Preferably, PCR is carried out using a DNA polymerase such as Pfu DNA polymerase, Taq DNA polymerase or an equivalent thermostable DNA polymerase. However, depending upon which cleavage reaction is used will dictate which polymerase is most appropriate. The annealing temperature of the PCR is about 5° C.-10° C. below the melting temperature of the labeled oligonucleotide pair.

The sequence of the first portion of the nucleic acid primer (target binding sequence) is designed such that hybridization to target DNA occurs at the annealing/extension temperature of a PCR reaction. Therefore, the sequence of the first sequence of the probe shares homology with the target DNA, whereas the second region of the nucleic acid probe, shares no homology to the target sequence. The sequence of the second portion of the nucleic acid primer (nucleic acid probe binding sequence) is designed such that hybridization to the nucleic acid probe occurs at the annealing/extension temperature of a PCR reaction.

The labeled oligonucleotide pair is subject to denaturation at appropriate conditions, including high temperatures, reduced ionic concentrations, and/or the presence of disruptive chemical agents such as formamide or DMSO. The nucleic acid probe and primer of the present invention preferably form a complex at the annealing/extension temperature, which is typically between 55-65° C. Therefore, a labeled oligonucleotide pair with a Tₘ higher than the annealing/extension temperature are preferred, and can have a Tₘ≧55° C., typically with a Tₘ≧60° C., Tₘ≧62° C., or Tₘ24 65° C., can be used. However, Tₘ generally should not be more than about 15° C. higher than the annealing/extension temperature. Most preferred are labeled oligonucleotide pairs with Tₘ in the range from about the annealing/extension temperature to about 10-15° C. above the annealing/extension temperature.

Kits

The invention is intended to provide novel compositions and methods for PCR as described herein. The invention herein also contemplates a kit format which comprises a package unit having one or more containers of the subject composition and in some embodiments including containers of various reagents used for polynucleotide synthesis, including synthesis in PCR. The kit may also contain one or more of the following items: polymerization enzymes (i.e., one or more nucleic acid polymerase, such as a DNA polymerase, especially a thermostable DNA polymerase), polynucleotide precursors (e.g., nucleoside triphosphates), primers, buffers, instructions, and controls. The kits may include containers of reagents mixed together in suitable proportions for performing the methods in accordance with the invention. Reagent containers preferably contain reagents in unit quantities that obviate measuring steps when performing the subject methods. One kit according to the invention also contains a DNA yield standard for the quantitation of the PCR product yields from a stained gel.

### EXAMPLES

The examples below are non-limiting and are merely representative of various aspects and features of the subject invention.

### Example 1

### Use of the Labeled Oligonucleotide Pair to Quantify CFTR Target DNA

A 100 fG of CFTR PCR product was used as template for all testing. 100 nM or 200 nM of the nucleic acid primer quenched with 5′-BHQ-2 was added to a FullVelocity# PCR reaction containing 400 nM CFTR forward primer and 200 nM of the nucleic acid probe labeled with 3′-FAM. FullVelocity# QPCR Master Mix is Stratagene Catalog No. 600561 and is described further in U.S. Pat. Nos. 6,528,254 and 6,548,250 (each incorporated herein by reference in their entirety).

The labeled oligonucleotide complex is depicted in FIG. 1. The nucleic acid sequence for the nucleic acid primer was

(SEQ ID NO:1)

5′-AGGGTTGCGATGGTTCTGTTGTAGGTAGGTTTGGTTGACTTGGTAG
G-3′

with the tag specific sequence (second portion) shown underlined. The nucleic acid sequence for the nucleic acid probe which was homologous to the tag (second portion) of the nucleic acid primer was

(SEQ ID NO:2)

5′ TACCTACAACAGAACCATCGCAACCCT-3′.

The nucleic acid sequence for the forward primer was

(SEQ ID NO:3)

5′ GCAGTGGGCTGTAAACTCC-3′.

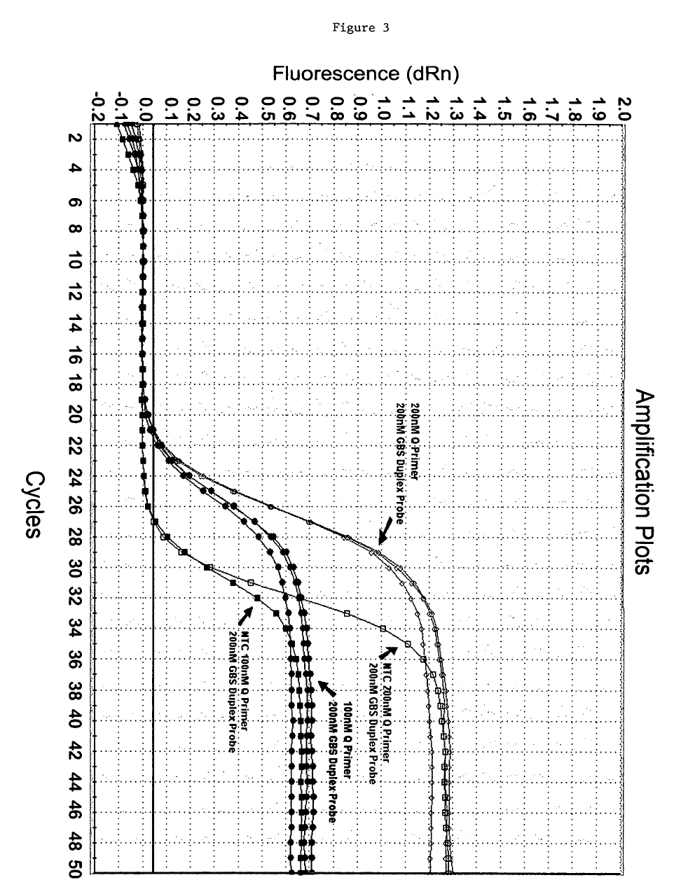

The experiment was conducted on an Mx300p real-time PCR instrument (Stratagene) with the following cycling parameters: 2 min at 95° C., followed by 50 cycles of 30 sec at 95° C., 30 sec at 60° C. Data is shown in FIG. 3. Data are expressed as dRn (change in FAM fluorescence, normalized to the reference dye) with respect to cycle number. The results showed a strong fluorescent signal with both the 100 nM and 200 nM nucleic acid primer. However, the final fluorescent signal was higher with the 200 nM concentration of the nucleic acid primer. Both reactions have the same Ct value

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims.

We claim:

1. A labeled oligonucleotide pair, comprising:
(a) a nucleic acid primer comprising a first portion and a second portion, wherein said first portion is complementary to a target nucleic acid and said second portion is complementary to a nucleic acid probe and is not complementary to said target nucleic acid;
(b) a nucleic acid probe comprising a portion complementary to said second portion of said nucleic acid primer, but not comprising a portion complementary to said first portion of said nucleic acid primer; and
(c) a pair of interactive labels, wherein a first member of said pair of interactive labels is coupled to said nucleic acid primer and a second member of said interactive pair of labels is coupled to said nucleic acid probe, wherein said primer and said probe form a hybrid, said labels interact and when said primer and probe are dissociated, said labels do not interact.

2. The labeled oligonucleotide pair of claim 1, wherein said pair of interactive labels comprises a fluorophore and a quencher.

3. The labeled oligonucleotide pair of claim 2, wherein one of said fluorophore or said quencher is attached to said 3′ nucleotide of said nucleic acid probe.

4. The labeled oligonucleotide pair of claim 3, wherein said fluorophore is attached to said 3′ nucleotide.

5. The labeled oligonucleotide pair of claim 3, wherein said quencher is attached to said 3′ nucleotide.

6. The labeled oligonucleotide pair of claim 2, wherein one of said fluorophore or said quencher is attached to said 5′ nucleotide of said nucleic acid primer.

7. The labeled oligonucleotide pair of claim 6, wherein said fluorophore is attached to said 5′ nucleotide.

8. The labeled oligonucleotide pair of claim 6, wherein said quencher is attached to said 5′nucleotide.

9. The labeled oligonucleotide pair of claim 6, wherein said fluorophore is attached to the 3′ terminal nucleotide of the nucleic acid probe and said quencher is attached to the 5′ terminal nucleotide of the nucleic acid primer.

10. The labeled oligonucleotide pair of claim 2, wherein said fluorophore is selected from the group consisting of FAM, R110, TAMRA, R6G, CAL Fluor Red 610, CAL Fluor Gold 540, and CAL Fluor Orange 560[ADD QUASAR-670].

11. The labeled oligonucleotide pair of claim 2, wherein said quencher is selected from the group consisting of DABCYL, BHQ-1, BHQ-2, and BHQ-3.

12. The labeled oligonucleotide pair of claim 2, wherein said fluorescence increases upon cleavage of said nucleic acid probe by at least 3 fold.

13. The probe of claim 2, wherein said fluorescence increases upon cleavage of said nucleic acid probe by at least 4 fold.

14. A kit comprising the labeled oligonucleotide pair of claim 1 and packaging material therefor, wherein said nucleic acid primer and said nucleic acid probe are in separate containers.

15. A kit, comprising the labeled oligonucleotide pair of claim 1 and packaging material therefor, where said nucleic acid primer and said nucleic acid probe are in the same container.

16. The kit of claim 14 or 15, further comprising a nucleic acid polymerase

17. The kit of claim 16, wherein said nucleic acid polymerase substantially lacks 5′ to 3′ nuclease activity.

18. The kit of claim 17, further comprising a nuclease.

19. The kit of claim 18, wherein said nuclease is an exonuclease.

20. The kit of claim 18, wherein said nuclease is an endonuclease.

21. The kit of claim 18, wherein said nuclease is a FEN nuclease.

22. A method of detecting a target nucleic acid in a sample, said method comprising:
(a) providing to a PCR reaction mixture the labeled oligonucleotide pair of claim 1; and
(b) permitting the cleavage of said nucleic acid probe, when said nucleic acid primer is hybridized to said target nucleic acid, so as to generate a detectable signal, wherein said signal is indicative of the presence of said target nucleic acid in the nucleic acid sample.

23. The method of claim 22, further comprising providing a nucleic acid polymerase

24. The method of claim 23, wherein said nucleic acid polymerase substantially lacks 5′ to 3′ nuclease activity.

25. The method of claim 24, further comprising a nuclease.

26. The method of claim 25, wherein said nuclease is an exonuclease.

27. The method of claim 25, wherein said nuclease is a FEN nuclease.

28. A method of detecting a target nucleic acid in a sample, said method comprising:
(a) performing a PCR amplification reaction, wherein said PCR amplification reaction mixture comprises a target nucleic acid, the labeled oligonucleotide pair of claim 1 and a second primer complementary to said target nucleic acid;
(b) performing a nuclease cleavage reaction; and
(c) detecting a signal generated by a member of said pair of interactive labels, wherein said signal is indicative of the presence of the target nucleic acid.

29. A reaction mixture comprising the probe of claim 1.
